(19) United States
(12) Patent Application Publication
(10) Pub. No.: US 2014/0038883 A1
(43) Pub. Date: Feb. 6, 2014

(54) NOVEL AZACOUMARIN DERIVATIVES HAVING MDR PUMP INHIBITING ACTIVITY

(71) Applicants: Anne Doleans-Jordheim, Saint Priest (FR); Jean Freney, Fleurieux sur I'Arbresle (FR); Charles Dumontet, Venissieux (FR); Ahcene Boumend Jel, Meylan (FR); Jean-Baptiste Veron, Le Mans (FR); Yung-Sing Wong, Saint Martin D'Heres (FR)

(72) Inventors: Anne Doleans-Jordheim, Saint Priest (FR); Jean Freney, Fleurieux sur I'Arbresle (FR); Charles Dumontet, Venissieux (FR); Ahcene Boumend Jel, Meylan (FR); Jean-Baptiste Veron, Le Mans (FR); Yung-Sing Wong, Saint Martin D'Heres (FR)

(73) Assignees: UNIVERSITE CLAUDE BERNARD LYON 1, Villeurbanne Cedex (FR); UNIVERSITE JOSEPH FOURIER, Grenoble (FR); CENTRE NATIONAL DE LA RECHERCHE SCIENTIFIQUE, Paris (FR); HOSPICES CIVIL DE LYON, Lyon Cedex 02 (FR)

(21) Appl. No.: 13/981,682

(22) PCT Filed: Jan. 27, 2012
(86) PCT No.: PCT/FR2012/050174

§ 371 Date: Oct. 15, 2013

(30) Foreign Application Priority Data
Jan. 28, 2011 (FR) 1150674

(51) Int. Cl. A61K 31/4704; A61K 38/14; A61K 31/7048; A61K 31/65; A61K 31/431; C07D 409/04; A61K 31/473; C07D 215/58; A61K 31/4709; A61K 45/06; C07D 401/04; C12Q 1/02; A61K 31/496

(52) U.S. Cl. CPC A61K 31/4704; C12Q 1/025; A61K 38/14; A61K 31/7048; A61K 31/65; A61K 31/431; A61K 31/496; A61K 31/473; C07D 215/58; A61K 31/4709; A61K 45/06; C07D 401/04; C07D 409/04
USPC 514/2.7; 435/32; 514/29; 514/154; 514/196; 514/253.08; 514/290; 514/312; 546/79; 546/154; 546/157

(57) ABSTRACT

The present invention relates to compounds of formula (I), where R₁ and R₂, identical or different, are each independently a hydrogen atom or a non-substituted or substituted (C₁-C₁₂) alkyl group; R₃ is a hydrogen atom or a non-substituted or substituted (C₁-C₆) alkyl group; R₄ is a non-substituted or substituted (C₁-C₁₂) alkyl group or an aryl or heteroaryl group, said aryl and heteroaryl groups being non-substituted or substituted; and R₅ is a non-substituted or substituted (C₁-C₁₂) alkyl group; or R₄ and R₅ are bonded to one another by a saturated hydrocarbon chain having 3 or 4 carbon atoms, optionally in hydrated form or in the form of a salt that is acceptable for being administered to animals or plants, for the use thereof as a potentiator of the effect of an antimicrobial agent or for the use thereof as an antimicrobial agent.

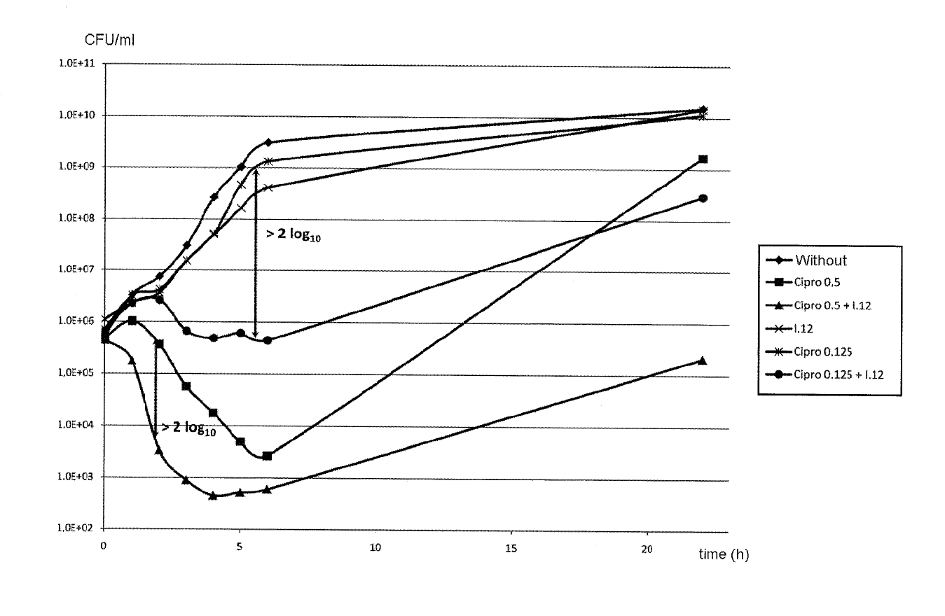

The subject of the present invention is novel azacoumarin derivatives having inhibitory activity on bacterial efflux pumps (EPI activity), in particular on the NorA pump, responsible for antibiotic resistance of MDR (Multidrug Resistance) type, and also such compositions for use thereof for increasing the effect of an antimicrobial agent or for use thereof as an antimicrobial agent.

Over the past few years, new bacterial strains exhibiting varied phenotypes of resistance to various antibiotic agents have developed. Because of the emergence of these resistances, a large number of these anti-infective molecules are now ineffective.

These resistance phenomena can affect all antibiotics (irrespective of their class and their chemical structure), and also their entire field of application, posing, as a result, serious public health problems. The consequences of these resistances are in fact major, whether from a medical, social or economical point of view (therapeutic blind alleys, increase in morbidity and mortality, longer periods of hospitalization, use of molecules which are expensive and/or which have harmful adverse effects, etc.).

Some of these resistances, identified in several gram-positive and gram-negative bacteria, in particular those involved in infections termed nosocomial (acquired in hospital), are linked to antibiotic efflux systems. The consequence of these effluxes is a decrease in the intracellular concentrations of antimicrobial agents and therefore in their efficacy (Efflux-mediated resistance to fluoroquinolones in gram-positive bacteria and the mycobacteria: Keith Poole; Antimicrobial Agents and Chemotherapy 2000, 44(10), 2595-2599). The antibiotics to which this type of resistance relates belong, for example, in the fluoroquinolone, tetracycline or macrolide classes. It should be noted that this type of resistance is also manifested with respect to certain antiparasitic and antitumor agents.

Bacterial efflux pumps are active transmembrane protein transporters which operate by virtue of the energy generated by the electrochemical gradient of the cytoplasmic membrane (Microbiological Reviews, 1996, 575-608) or by ATP hydrolysis. For further details, reference may be made to: Journal of Antimicrobial Chemotherapy, 2003, 51, 9-11, Antimicrobial Agents and Chemotherapy 2000, 44(9), 2233-2241, Molecular Microbiology 2000, 36(3), 772-773 and Current opinion in drug discovery & development 2001, 4(2), 237-45. The function of these systems is identical despite their structural diversity and their source of energy: they oppose the intracellular accumulation of their substrates, such as heavy metals, bile salts and, in the present case, antibiotics. As previously mentioned, these systems have a negative impact in antibiotic therapy since they can (i) partially reduce the antibiotic's own activity, (ii) potentiate the effect of other pre-existing mechanisms of resistance (enzymatic inactivation of the antibiotic or modification of its target, impairment of bacterial membrane permeability, etc.), (iii) promote the emergence of bacteria resistant to conventional antibiotics following genetic mutations (for example, fluoroquinolone gyrases), (iv) generally, facilitate the adaptation and persistence of bacteria in vivo.

The use of bacterial efflux pump inhibitors constitutes one of the solutions that can be envisaged for combating bacterial resistances linked to antibiotic efflux (Journal of Medicinal Chemistry 2001, 44(2), 261-268, Antimicrobial agents and chemotherapy 2001, 45(1), 105-16, Antimicrobial agents and chemotherapy 1999, 43, 2404-2408 and Antimicrobial agents and chemotherapy 2003, 47 (2), 719-726)). The microorganisms to which these inhibitors relate are bacteria that are resistant to antibiotics via a mechanism of efflux, in particular staphylococci such as Staphylococcus aureus, streptococci such as Streptococcus pneumoniae, enterococcus such as Enterococcus faecalis or E. faecium, or other bacterial species, including Bacillus subtilis, Escherichia coli, Pseudomonas aeruginosa, Haemophilus influenzae, etc. Various pumps can be targeted, including the NorA pump, responsible for the expulsion of hydrophilic fluoroquinolones (norfloxacin and/or ciprofloxacin).

Efflux pump inhibitors, and in particular NorA pump inhibitors, could thus have an important place in therapy by being used in particular in combination with various antimicrobial agents such as antibiotics or antiseptics. These inhibitors could restore activity to antibiotics which have become ineffective on multiresistant bacteria, by increasing their intracellular concentration. They could also make safe the use of some other antibiotics by considerably reducing the emergence of resistances, in particular through the appearance of mutations in their targets. It should be noted that competitive inhibitors should be active on a large number of microorganism species, given the relative substrate-specificity of the pumps and the homologies between the various transporters.

Some efflux pump inhibitors are described in the literature. Mention may, for example, be made of the quinolone derivatives described in documents U.S. Pat. No. 6,346,391, U.S. Pat. No. 6,271,416, US 2007/078176 and US 2003/149074, or else the derivatives comprising a thiophene or benzothiophene group as described in application WO 2006/018544.

In this context, the inventors have identified novel efflux pump inhibitors, in particular NorA pump inhibitors. The inventors have demonstrated that these compounds, of azacoumarin-type structure, are capable of restoring the activity of a class of customary antibiotics of the fluoroquinolone family with respect to resistant bacterial strains. These inhibitors, used in compositions, in particular pharmaceutical compositions, would improve the action of an antibiotic of which the efficacy has been reduced, owing to its expulsion by the NorA pump. These inhibitors can also be used in diagnostic tests intended for identifying strains expressing the resistance phenotype. Using a biological sample taken from an infected patient, the minimum inhibitory concentrations (MICs) of the antibiotic used in the treatment (preferably a fluoroquinolone, including ciprofloxacin) could be determined in the presence or absence of one of these inhibitors. The results obtained should provide information on the existence and the nature of a mechanism of resistance to be taken into account in the treatment.

More specifically, the subject of the present invention is compounds of formula (I):

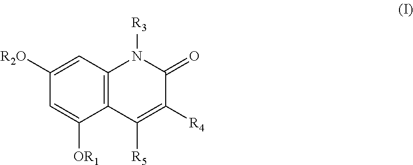

in which:
R₁ and R₂, which may be identical or different, are each independently a hydrogen atom or an unsubstituted or substituted (C₁-C₁₂)alkyl group,
R₃ is a hydrogen atom or an unsubstituted or substituted (C₁-C₆)alkyl group, or an unsubstituted or substituted benzyl group,
R₄ is an unsubstituted or substituted (C₁-C₁₂)alkyl group, an aryl group or a heteroaryl group, it being possible for said aryl and heteroaryl groups to be unsubstituted or substituted, and R₅ is an unsubstituted or substituted (C₁-C₁₂)alkyl group,
or else R₄ and R₅ are linked to one another by a saturated hydrocarbon chain containing 3 or 4 carbon atoms,

optionally in hydrated form or in the form of a salt which is acceptable for administration to animals or plants,

for use thereof as a potentiator of the effect of an antimicrobial agent or for use thereof as an antimicrobial agent.

According to particular embodiments, the compounds of formula (I) according to the invention have one or more, or even all, of the characteristics below:
R₃ is a hydrogen atom, or a methyl or benzyl group,
R₁ and R₂, which may be identical or different, are each independently a hydrogen atom or a methyl group,
R₅ is a methyl group,
R₄ is a benzyl, phenyl, naphthyl, thiophenyl and indolyl group, it being possible for said groups to be unsubstituted or substituted with one or more substituents chosen from chlorine, bromine, iodine and fluorine atoms, and (C₁-C₆)alkyl and (C₁-C₆)alkoxy groups.

According to preferred embodiments resulting in compounds having an appropriate antibiotic effect, the compounds of formula (I) according to the invention have one or more, or even all, of the characteristics below:
R₃ is a hydrogen atom,
R₄ is an unsubstituted phenyl group, an unsubstituted heteroaryl (and in particular thiophenyl or indolyl) group or a phenyl group bearing one, two, three or four substituents chosen from chlorine, bromine, iodine and fluorine atoms, and (C₁-C₆)alkyl and (C₁-C₆)alkoxy groups, chlorine or methoxy substituents being preferred,
R₅ is a methyl group,
R₁ is a hydrogen atom,
R₂ is a (C₁-C₆)alkyl group, and in particular a methyl group.

According to other preferred embodiments, resulting in compounds having a particularly high NorA-efflux pump-inhibiting activity, the compounds of formula (I) according to the invention have one or more, or even all, of the characteristics below:
R₃ is a hydrogen group, or a (C₁-C₆)alkyl group and in particular a methyl group,
R₄ is an aryl group, an aryl(C₁-C₆)alkyl group, and in particular a benzyl group, or a heteroaryl group,
R₅ is a (C₁-C₆)alkyl group, and in particular a methyl group,
R₁ is a methyl group,
R₂ is a (C₁-C₆)alkyl group, and in particular a methyl group.

By way of examples of compounds according to the invention, mention may be made of:
3-(3-chlorophenyl)-5,7-dimethoxy-4-methylquinolin-2(1H)-one, compound I.1,
5,7-dimethoxy-3-(4-methoxyphenyl)-4-methylquinolin-2(1H)-one, compound I.2,
5,7-dimethoxy-4-methyl-3-(1-methyl-1H-indol-3-yl)quinolin-2(1H)-one, compound I.3,
5,7-dimethoxy-4-methyl-3-(thiophen-2-yl)quinolin-2(1H)-one, compound I.4,
5,7-dimethoxy-4-methyl-3-phenylquinolin-2(1H)-one, compound I.5,
3-(1H-indol-3-yl)-5,7-dimethoxy-4-methylquinolin-2(1H)-one, compound I.6,
5-hydroxy-7-methoxy-4-methyl-3-(thiophen-2-yl)quinolin-2(1H)-one, compound I.7,
5-hydroxy-7-methoxy-1,4-dimethyl-3-phenylquinolin-2(1H)-one, compound I.8,
5-hydroxy-7-methoxy-4-methyl-3-(naphthalen-2-yl)quinolin-2(1H)-one, compound I.9,
5,7-dimethoxy-1,4-dimethyl-3-phenylquinolin-2(1H)-one, compound I.10,
7-hydroxy-5-methoxy-4-methyl-3-phenylquinolin-2(1H)-one, compound I.11,
5-hydroxy-7-methoxy-4-methyl-3-phenylquinolin-2(1H)-one, compound I.12,
5,7-dihydroxy-4-methyl-3-phenylquinolin-2(1H)-one, compound I.13,
3-benzyl-5-hydroxy-7-methoxy-4-methylquinolin-2(1H)-one, compound I.14,
2,3-dihydro-9-hydroxy-7-methoxy-1H-cyclopenta[c]quinolin-4(5H)-one, compound I.15,
1-benzyl-5,7-dimethoxy-4-methyl-3-phenylquinolin-2(1H)-one, compound I.16.

The subject of the present invention is also the compounds I.1 to I.16 as such, and also the compounds as such of formula (Ip):

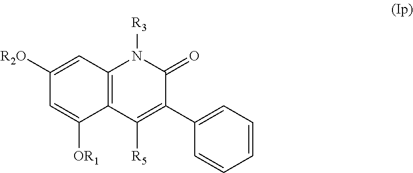

in which:
R₁ and R₂, which may be identical or different, are each independently a hydrogen atom or an unsubstituted or substituted (C₁-C₁₂)alkyl group,
R₃ is a hydrogen atom or an unsubstituted or substituted (C₁-C₆)alkyl group, or an unsubstituted or substituted benzyl group,
R₅ is an unsubstituted or substituted (C₁-C₁₂)alkyl group,
   optionally in hydrated form or in the form of a salt which is acceptable for administration to animals or plants.

According to particular embodiments, the compounds of formula (Ip) according to the invention have one or more, or even all, of the characteristics below:
R₅ is a methyl group,
R₃ is a hydrogen atom or a methyl or benzyl group,
R₁ and R₂, which may be identical or different, are each independently a hydrogen atom or a methyl group.

Among these compounds of formula (Ip), those in which R₅=Me, R₁=H and R₂=Me are particularly preferred.

The description hereinafter makes it possible to understand the invention more clearly. By way of introduction, a certain number of definitions are recalled.

The term alkyl is intended to mean, when not otherwise specified, a linear or branched, saturated hydrocarbonated radical. The term “(C₁-C₆)alkyl group is intended to mean an alkyl group which comprises from 1 to 6 carbon atoms. By way of examples of an alkyl group, mention may be made of methyl, ethyl, n-propyl, isopropyl, n-butyl, isobutyl, sec-butyl, t-butyl, n-pentyl and n-hexyl groups.

The term aryl group is intended to mean a monocyclic, bicyclic or polycyclic carbocycle preferably comprising from 6 to 12 carbon atoms, comprising at least one aromatic group, for example a phenyl, cinnamyl or naphthyl group. Phenyl is the aryl group which is particularly preferred.

The term heteroaryl group is intended to mean a monocyclic, bicyclic or polycyclic carbocycle preferably comprising from 6 to 12 carbon atoms, and comprising at least one heteroatom and aromatic group. By way of example of a heteroaryl group, mention may be made of thiophenyl, indolyl, pyridyl, benzopheranyl and benzothiophenyl groups.

The term substituted group is intended to mean a group which is monosubstituted or polysubstituted with two or more identical or different substituents chosen from: a fluorine, chlorine, bromine or iodine atom, a hydroxyl, (C₁-C₁₂)alkyl, (C₁-C₁₂)alkenyl, (C₁-C₁₂)alkoxy, (C₅-C₁₂)cycloalkyl, benzyloxy, aryl, sulfhydryl or carboxy group, or an amine group —NRₐR_{b} with Rₐ and R_{b}, which may be identical or different, each being, independently of one another, a hydrogen atom or a (C₁-C₁₂)alkyl group or else Rₐ and R_{b} are linked to one another so as to form, with the nitrogen atom to which they are bonded, a piperidine, a pyrrolidine, a piperazine, an N—(C₁-C₁₂)alkylpiperazine or a morpholine. The benzyl group is an example of a substituted alkyl group.

The term alkenyl corresponds to an alkyl group as defined above, comprising a double bond. The vinyl, allyl, isopropenyl, 1-, 2- or 3-butenyl, pentenyl and hexenyl groups are examples of such alkenyl groups.

The term alkoxy denotes an O-alkyl group.

The term cycloalkyl denotes a cycloalkyl group comprising from 3 to 10 carbon atoms, for example cyclopropyl, cyclobutyl, cyclopentyl or cyclohexyl, bridged cycloalkyl groups such as adamantyl or bicyclo[3.2.1]octanyl groups. The term heterocycloalkyl group denotes a cycloalkyl group as defined above, comprising one or more heteroatoms, selected from nitrogen, oxygen and sulfur atoms.

The term sulfhydryl denotes —SH and the term carboxyl denotes —COOH.

The term “treatment” denotes any therapeutic measure which is prophylactic or which suppresses a disease or disorder resulting in a desirable clinical effect or in any beneficial effect, including in particular the suppression or the reduction of one or more symptoms, or the regression, slowing down or cessation of the progression of the disorder which is associated therewith.

The expression “therapeutically effective amount” denotes any amount of a composition which improves one or more of the characteristic parameters of the affection treated.

The expression “potentiator of the effect of an antimicrobial agent” is intended to mean that the antimicrobial effect of an antimicrobial agent is increased, when the latter is used in combination with the potentiating agent, namely a compound of formula (I) or (Ip). This increase may in particular be demonstrated in one of the tests presented in the examples hereinafter. In particular, the antimicrobial effect then observed is increased by at least a factor of 4 compared with the reference activity obtained with the antimicrobial agent tested in the absence of potentiating agent, namely the compound of formula (I) or (Ip), which means that the minimum inhibiting concentration of the antimicrobial agent then obtained is at least divided by 4 compared with that required in the absence of potentiating agent.

The term “antimicrobial agent” is intended to mean in particular substances capable of inhibiting the growth of microorganisms, or even of killing them. In the context of the invention antibiotics having a bacteriostatic activity (substance inhibiting bacterial growth) and bactericidal activity (substance which kills bacteria) are targeted. More particularly, the EPI activity of the molecules favors the fluoroquinolone class, including ciprofloxacin.

The compounds used in the context of the invention are prepared according to conventional techniques. For example, the compounds of formula (I) can be obtained according to SCHEME 1 below, in which R₃, R₄ and R₅ are as defined previously for (I), R′₁ and R′₂ are respectively R₁ and R₂ as defined previously for (I) or a group which is a precursor of the latter, X is a halogen atom and in particular a chlorine atom, and R′ is an alkyl group and in particular an ethyl group.

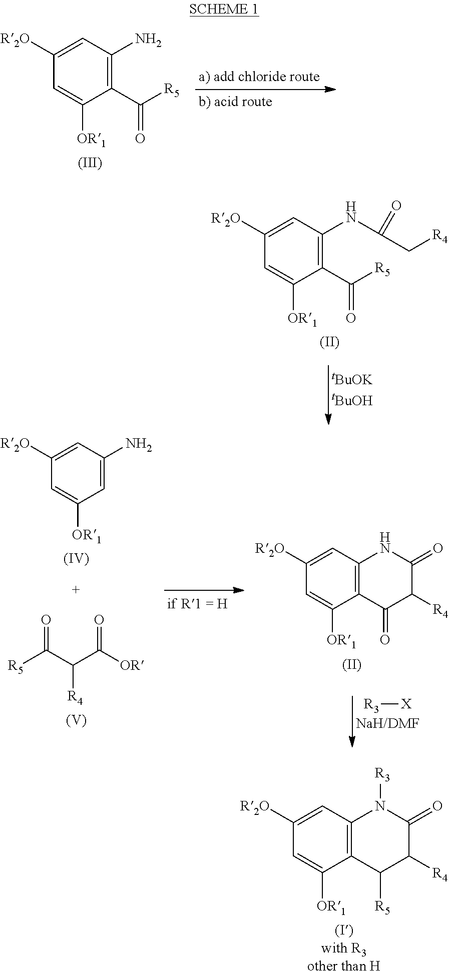

The compounds of formula (I′) in which R₃=H can in particular be obtained from a compound of formula (II), under the action of a tBuOK/tBuOH mixture. The compound of formula (II) can, for its part, be obtained from the compound of formula (III) which is commercial or obtained according to simple chemical syntheses, either by reacting an acid chloride R₄—CH₂—C(O)—Cl, in the presence of triethylamine, or by reacting an acid R₄—CH₂—C(O)—OH, in the presence of triethylamine and of an acid such as bis(2-oxo-3-oxazolidinyl)phosphonic acid (BOP-Cl).

Alternatively, when R′₁=H, the compounds of formula (I′) in which R₃=H can be prepared from the phenols (IV), by coupling with a compound (V) under hot conditions in a solvent such as chlorobenzene.

Next, the compounds of formula (I′) in which R₃ is other than H can be prepared from the corresponding compound of formula (I′) in which R₃=H, by alkylation of the amine function by reacting a halide, and in particular an alkyl chloride X—R₃, in the presence of a hydride such as NaH in DMF.

The compounds of formula (I′) thus prepared can correspond to a compound of formula (I), if the R′₁ and R′₂ groups are respectively R₁ and R₂. It is also possible to obtain the desired R₁ or R₂ group from a precursor group R′₁ or R′₂ after one or more conversions. For example, a group R₁=H may be obtained by reduction of a group R₁=Me, by reacting BBr₃ in dichloromethane.

The molecules of formula (I) can therefore be accessed by simple chemistry, and can be obtained at a low preparation cost.

The salts of the compounds according to the invention are prepared according to techniques well known to those skilled in the art. The salts of the compounds of formulae (I) and (Ip) according to the present invention comprise those with inorganic or organic acids or bases which enable suitable separation or crystallization of the compounds of formula (I) or (Ip), and also pharmaceutically acceptable salts. As appropriate acid, mention may be made of: oxalic acid or an optically active acid, for example a tartaric acid, a dibenzoyltartaric acid, a mandelic acid or a camphosulfonic acid, and those which form physiologically acceptable salts, such as the hydrochloride, hydrobromate, sulfate, hydrogen sulfate, dihydrogen phosphate, maleate, fumarate, 2-naphthalenesulfonate, para-toluenesulfonate, mesylate, besylate or isothionate. As appropriate base, mention may be made of: lysine, arginine, meglumine, benethamine, benzathine and those which form physiologically acceptable salts, such as sodium, potassium or calcium salts.

As compound in hydrated form, mention may be made, by way of example, of hemihydrates, monohydrates and polyhydrates.

Compounds of formulae (I) and (Ip) above also comprise those in which one or more hydrogen, carbon or halogen atoms, in particular chlorine or fluorine atoms, have been replaced with their radioactive isotope, for example tritium or carbon-14. Such labeled compounds are of use in research, metabolism or pharmacokinetic studies, or in biochemical assays.

The functional groups optionally present in the molecule of the compounds of formula (I) or (Ip) and in the reaction intermediates can be protected, either in a permanent form or in a temporary form, by protective groups which ensure unambiguous synthesis of the expected compounds. The protection and deprotection reactions are carried out according to techniques well known to those skilled in the art. The expression “temporary protective group for amines, alcohols or carboxylic acids” is intended to mean protective groups such as those described in Protective Groups in Organic Synthesis, Greene T. W. and Wuts P. G. M., published by John Wiley and Sons, 2006 and in Protecting Groups, Kocienski P. J, 1994, Georg Thieme Verlag.

The inventors have demonstrated the potentiating effect of the compounds according to the invention using fluoroquinolones, and in particular hydrophilic fluoroquinolones such as norfloxacin or ciprofloxacin, on gram-positive bacteria belonging to the staphylococcus or enterococcus genus. These effects relate in particular to resistant strains, such as methicillin-resistant Staphylococcus aureus (MRSA) or glycopeptide-resistant Staphylococcus aureus (GISA for glycopeptides-intermediate S. aureus). In general, the compounds according to the invention exhibit an activity which improves by a factor of 2 to 128 the activity of the conventional antibiotic. The mechanism of action of this potentiating effect involves an inhibitory activity on bacterial efflux pumps, and in particular on NorA pumps.

The compounds according to the invention can therefore be used for potentiating the effect, i.e. increasing the effect, of antimicrobial agents and in particular of antibiotics which have become inactive on strains which are resistant via an efflux mechanism. The term “potentiation” is intended to mean that, when a compound of formula (I) or (Ip) is combined with an antimicrobial agent, an antimicrobial effect is obtained which is greater than that obtained with either of the compounds, and even synergistic, i.e. greater than the sum of the effects obtained separately.

The compounds of formula (I) or (Ip) can thus be used for restoring the action of conventional antibiotics, in the case where efflux pumps are responsible for a significant resistance to these antibiotics. As an example of resistance to antibiotics, mention may be made of the resistance to quinolones of Staphylococcus aureus and of Streptococcus pneumoniae, and also the various resistances of Pseudomonas aeruginosa (ASM News, (1997), 63, 605-610). Reference may also be made to the Merck Index, 11th Ed., Budavari ed., 1989, Merck & Co., Inc., Rahway, N. 3., pp. THER-9 to THER-11 and THER-13, which describes a certain number of antibiotic agents, the effect of which can be potentiated by virtue of the compounds of formula (I) or (Ip).

In the light of these results, the compounds of formula (I) or (Ip) can be used in pharmaceutical or plant protection compositions, intended to restore the efficacy of antibiotic agents having been affected by a mechanism of expulsion via NorA. These compositions can contain, in addition to the inhibitor, an antibiotic, in particular of the fluoroquinolone family, and a usual excipient for the ingestion and the transport of the active ingredients.

Furthermore, no sign of cell toxicity has been observed with the compounds according to the invention at the pharmacologically active doses. Their toxicity is therefore compatible with their use as medicaments.

Generally, the compounds according to the invention may be used for preparing a medicament with antimicrobial activity or, preferably, for preparing a medicament intended for improving the action of antimicrobial agents, the efficacy of which is affected by efflux pump, in particular NorA, mechanisms. In the latter case, the administration of a compound of formula (I) or (Ip) is therefore accompanied by the administration of the antimicrobial agent of which it is desired to improve the activity. The compound of formula (I) or (Ip) can be formulated in combination with said antimicrobial agent or can be the subject of a separate formulation. It may also be used for carrying out diagnostic tests such as an antibiogram making it possible to demonstrate, for the strain concerned, a mechanism of resistance by efflux.

The subject of the present invention is therefore also the compounds of formula (Ip), and also the compounds I.1 to I.16, the pharmaceutically compatible salts thereof, or optionally the solvents or hydrates thereof, as medicaments.

The compositions administrable to plants and to animals (including human beings) contain an effective dose of a compound according to the invention or of an acceptable salt, solvate or hydrate thereof, and suitable excipients.

Said excipients are chosen according to the form and the mode of administration desired. In the pharmaceutical compositions of the present invention for oral, sublingual, subcutaneous, intramuscular, intravenous, topical, intratracheal, intranasal, transdermal, rectal or intraocular administration, the active ingredients of formula (I) or (Ip) above, or the optional salts, solvates and hydrates thereof, can be administered in unit administration forms, as a mixture with conventional pharmaceutical salts, to animals and to human beings for the prophylaxis or the treatment of infectious diseases linked to resistant or nonresistant bacteria. The appropriate unit administration forms include oral forms, such as tablets, gel capsules, powders, granules and oral solutions or suspensions, sublingual, buccal, intratracheal or intranasal administration forms, subcutaneous, intramuscular or intravenous administration forms and rectal administration forms. For topical application, the compounds according to the invention can be used in creams, ointments, lotions or eye lotions.

In order to obtain the effect, the dose of active ingredient preferably ranges between 1 and 100 mg per kg of body weight and per day. The compound (I) or (Ip) and the antimicrobial agent of which the effect is to be potentiated are advantageously administered in a ratio of 4 to 1.

When a solid composition in tablet form is prepared, the main active ingredient is mixed with a pharmaceutical vehicle, such as gelatin, starch, lactose, magnesium stearate, talc, gum Arabic, or the like. The tablets can be coated with sucrose, with a cellulose-based derivative, or with other suitable materials, or else they can be treated such that they have a sustained or delayed activity and that they continuously release a predetermined amount of active ingredient.

A preparation in gel capsules is obtained by mixing the active ingredient with a diluent and by pouring the mixture obtained into soft or hard gel capsules.

Pharmaceutical compositions containing a compound of the invention can also be in liquid form, for example solutions, emulsions, suspensions or syrups. The appropriate liquid supports may be water, organic solvents such as glycerol or glycols, and also mixtures thereof, in varied proportions, in water.

In preparation in syrup form, elixir form or for administration in the form of drops may contain the active ingredient together with a sweetener, preferably a calorie-free sweetener, methylparaben and propylparaben as antiseptic, and also a flavoring and a suitable colorant. The water-dispersible powders or granules can contain the active ingredient as a mixture with dispersants or wetting agents, or suspension agents, such as polyvinylpyrrolidone, and also with sweeteners or flavor enhancers.

The subject of the present invention is also pharmaceutical compositions containing several active ingredients in combination, one of which is a compound (I) or (Ip) and the other of which is an antimicrobial agent as previously defined.

Moreover, generally, the same preferences as those indicated previously for the compounds and compositions are applicable, mutatis mutandis, to the medicaments and uses employing these compounds.

The subject of the present invention is also the use of the inhibitors as defined above, in diagnostic methods and in particular the use thereof for demonstrating, in vitro, the presence in a biological sample of bacteria resistant to an antibiotic and also their degree of resistance. The syntheses and descriptions of the biological tests hereinafter, with reference to the appended figures, illustrate the invention without, however, limiting it.

### A. EXAMPLES

The following compounds presented in TABLE 1 were synthesized.

TABLE 1

| Compound | Structure | IUPAC Name |
|---|---|---|
| I.1 | 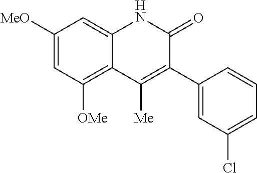 [SMILES: COc1cc(OC)c2c(C)c(-c3cccc(Cl)c3)c(=O)[nH]c2c1] | 3-(3-chlorophenyl)-5,7- dimethoxy-4-methylquinolin- 2(1H)-one |
| I.2 | 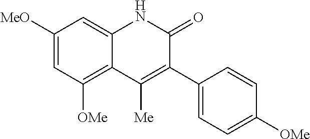 [SMILES: COc1ccc(-c2c(C)c3c(OC)cc(OC)cc3[nH]c2=O)cc1] | 5,7-dimethoxy-3-(4- methoxyphenyl)-4- methylquinolin-2(1H)-one |
| I.3 | 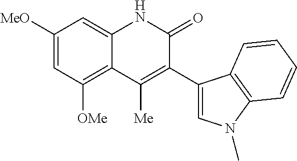 [SMILES: COc1cc(OC)c2c(C)c(-c3cn(C)c4ccccc34)c(=O)[nH]c2c1] | 5,7-dimethoxy-4-methyl-3-(1- methyl-1H-indol-3-yl)quinolin- 2(1H)-one |
| I.4 | 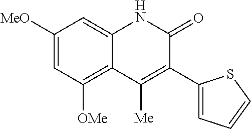 [SMILES: COc1cc(OC)c2c(C)c(-c3cccs3)c(=O)[nH]c2c1] | 5,7-dimethoxy-4-methyl-3- (thiophen-2-yl)quinolin-2(1H)-one |
| I.5 | 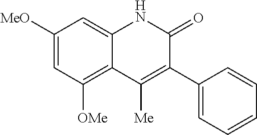 [SMILES: COc1cc(OC)c2c(C)c(-c3ccccc3)c(=O)[nH]c2c1] | 5,7-dimethoxy-4-methyl-3- phenylquinolin-2(1H)-one |
| I.6 | 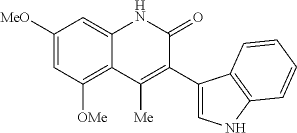 [SMILES: COc1cc(OC)c2c(C)c(-c3c[nH]c4ccccc34)c(=O)[nH]c2c1] | 3-(1H-indol-3-yl)-5,7-dimethoxy- 4-methylquinolin-2(1H)-one |
| I.7 | 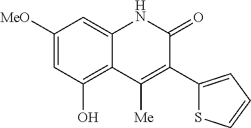 [SMILES: COc1cc(O)c2c(C)c(-c3cccs3)c(=O)[nH]c2c1] | 5-hydroxy-7-methoxy-4-methyl- 3-(thiophen-2-yl)quinolin-2(1H)- one |
| I.8 | 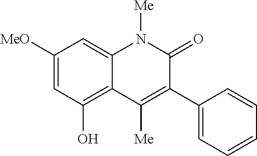 [SMILES: COc1cc(O)c2c(C)c(-c3ccccc3)c(=O)n(C)c2c1] | 5-hydroxy-7-methoxy-1,4- dimethyl-3-phenylquinolin-2(1H)- one |
| I.9 | 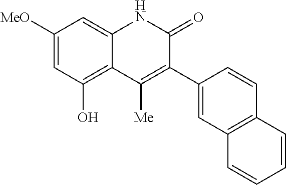 [SMILES: COc1cc(O)c2c(C)c(-c3ccc4ccccc4c3)c(=O)[nH]c2c1] | 5-hydroxy-7-methoxy-4-methyl- 3-(naphthalen-2-yl)quinolin- 2(1H)-one |
| I.10 | 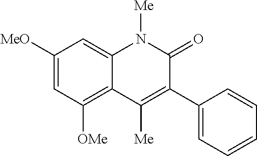 [SMILES: COc1cc(OC)c2c(C)c(-c3ccccc3)c(=O)n(C)c2c1] | 5,7-dimethoxy-1,4-dimethyl-3- phenylquinolin-2(1H)-one |
| I.11 | 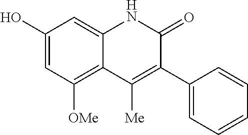 [SMILES: COc1cc(O)cc2[nH]c(=O)c(-c3ccccc3)c(C)c12] | 7-hydroxy-5-methoxy-4-methyl- 3-phenylquinolin-2(1H)-one |
| I.12 | 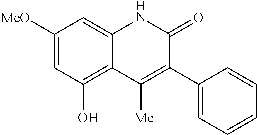 [SMILES: COc1cc(O)c2c(C)c(-c3ccccc3)c(=O)[nH]c2c1] | 5-hydroxy-7-methoxy-4-methyl- 3-phenylquinolin-2(1H)-one |
| I.13 | 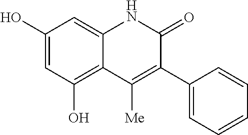 [SMILES: Cc1c(-c2ccccc2)c(=O)[nH]c2cc(O)cc(O)c12] | 5,7-dihydroxy-4-methyl-3- phenylquinolin-2(1H)-one |
| I.14 | 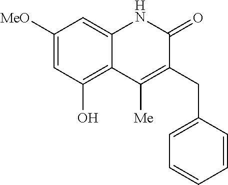 [SMILES: COc1cc(O)c2c(C)c(Cc3ccccc3)c(=O)[nH]c2c1] | 3-benzyl-5-hydroxy-7-methoxy-4- methylquinolin-2(1H)-one |
| I.15 | 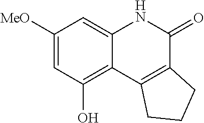 [SMILES: COc1cc(O)c2c3c(c(=O)[nH]c2c1)CCC3] | 2,3-dihydro-9-hydroxy-7- methoxy-1H-cyclopenta[c] quinolin-4(5H)-one |
| I.16 | 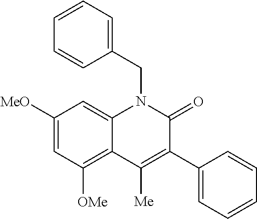 [SMILES: COc1cc(OC)c2c(C)c(-c3ccccc3)c(=O)n(Cc3ccccc3)c2c1] | 1-benzyl-5,7-dimethoxy-4- methyl-3-phenylquinolin-2(1H)- one |

Method A:

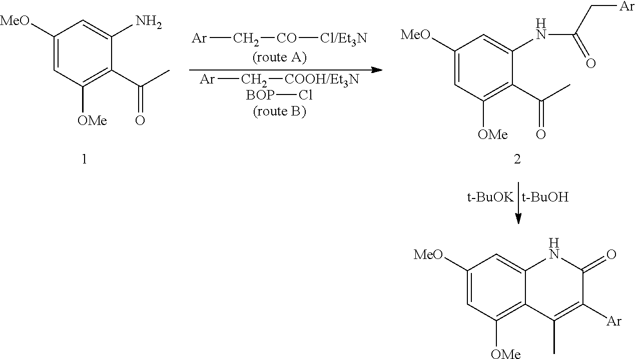

The compounds listed in TABLE 2 below were synthesized according to method A.

TABLE 2

| Compound | Ar = R₄ |
|---|---|
| I.1 | 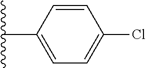 [SMILES: *c1ccc(Cl)cc1] |
| I.2 | 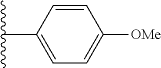 [SMILES: *c1ccc(OC)cc1] |
| I.3 | 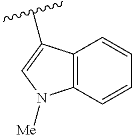 [SMILES: *c1cn(C)c2ccccc12] |
| I.4 | 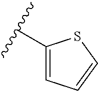 [SMILES: *c1cccs1] |
| I.5 | 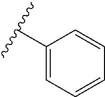 [SMILES: *c1ccccc1] |
| I.6 | 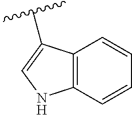 [SMILES: *c1c[nH]c2ccccc12] |

Method B

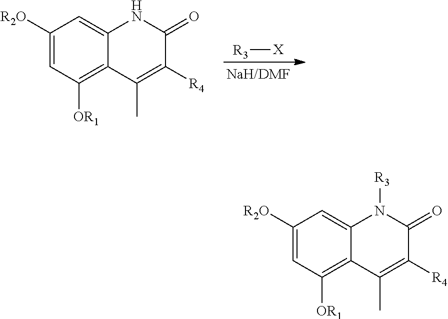

The compounds listed in TABLE 3 below were prepared according to the method B.

TABLE 3

| | |
|---|---|
| I.10 | 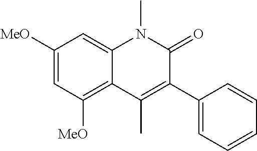 [SMILES: COc1cc(OC)c2c(C)c(-c3ccccc3)c(=O)n(C)c2c1] |
| I.16 | 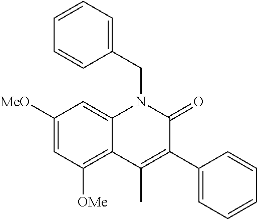 [SMILES: COc1cc(OC)c2c(C)c(-c3ccccc3)c(=O)n(Cc3ccccc3)c2c1] |

Method C:

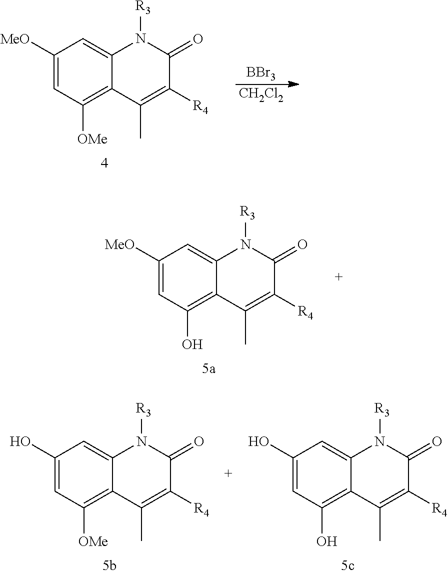

The compounds listed in TABLE 4 below were prepared according to method C.

TABLE 4

| | |
|---|---|
| I.7 | 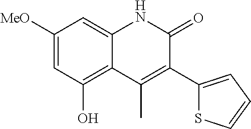 [SMILES: COc1cc(O)c2c(C)c(-c3cccs3)c(=O)[nH]c2c1] |
| I.9 | 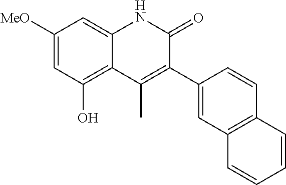 [SMILES: COc1cc(O)c2c(C)c(-c3ccc4ccccc4c3)c(=O)[nH]c2c1] |
| I.11 | 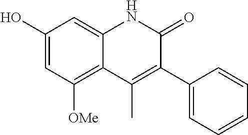 [SMILES: COc1cc(O)cc2[nH]c(=O)c(-c3ccccc3)c(C)c12] |
| I.13 | 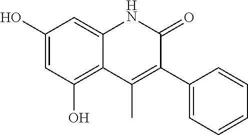 [SMILES: Cc1c(-c2ccccc2)c(=O)[nH]c2cc(O)cc(O)c12] |
| I.8 | 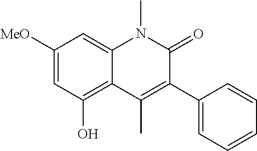 [SMILES: COc1cc(O)c2c(C)c(-c3ccccc3)c(=O)n(C)c2c1] |

Method D:

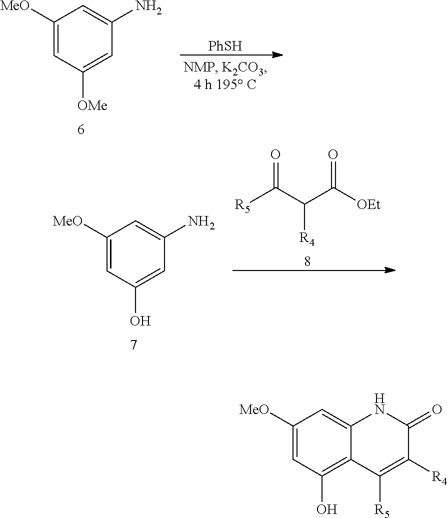

The compounds listed in TABLE 5 below were prepared according to method D.

TABLE 5

| | |
|---|---|
| I.12 | 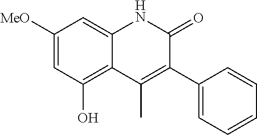 [SMILES: COc1cc(O)c2c(C)c(-c3ccccc3)c(=O)[nH]c2c1] |
| I.14 | 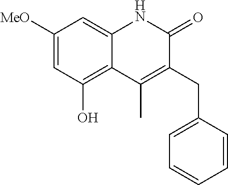 [SMILES: COc1cc(O)c2c(C)c(Cc3ccccc3)c(=O)[nH]c2c1] |
| I.15 | 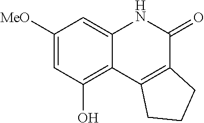 [SMILES: COc1cc(O)c2c3c(c(=O)[nH]c2c1)CCC3] |

Method A

First Step:

Route A:

The aniline derivative 1 (commercial or prepared according to Feka et al. Heterocycles, 2002, 57, 123-128) is dissolved in tetrahydrofuran (5 ml/mmol) at 0° C. and under argon. Triethylamine (1.2 eq) is added, followed by the dropwise addition of arylacetic chloride (1.2 eq.) in solution in tetrahydrofuran (9 ml/mmol). The reaction mixture is stirred for 15 h at ambient temperature and then hydrolyzed by adding H₂O. The solution is extracted with ethyl acetate and the organic phase is washed successively with a solution of NaHCO₃ (5% in water) and a saturated solution of NaCl. The organic phase is dried over MgSO₄ and then concentrated. Purification on a silica gel column eluted with CH₂Cl₂/MeOH (99.5:0.5; v:v) gives the pure product 2.

Route B:

The aniline derivative 2 is dissolved in DMF (8 ml/mmol) under an argon atmosphere and then treated successively with Et₃N (2 eq.), BOP-Cl (2 eq.) and 2-arylacetic acid (2 eq.). The reaction is stirred at ambient temperature for 48 h and then stopped by adding NaHCO₃ (5% in H₂O). The DMF is evaporated off under vacuum and the residue is extracted with ethyl acetate, washed with a saturated solution of NaCl, then dried over MgSO₄ and then concentrated. Purification on a silica gel column eluted with CH₂Cl₂ gives the expected product 2.

Second Step:

The derivative 2 (1.52 g, 4.39 mmol) is dissolved in t-BuOH (5 ml/mmol). t-BuOK (5 eq.) is added and the solution is stirred at ambient temperature for 12 hours. The t-BuOH is then evaporated off under vacuum and a saturated solution of NH₄Cl is added. The solution is extracted with ethyl acetate (EtOAc), washed with water, then with a saturated solution of NaCl and, finally, dried over MgSO₄. The organic phase is concentrated and the product is crystallized from MeOH, and then the crystals are washed with CH₂Cl₂ so as to give the pure product 3.

Method B

NaH (4 eq.) is added to a solution of the derivative 3 in THF (8 ml/mmol) and under an argon atmosphere and the solution is stirred at ambient temperature for 30 min. Methyl iodide (1.5 eq.) is added dropwise and the solution is left to stir for 24 h. The reaction is stopped by adding H₂O and then extracted with ethyl acetate. The organic phase is dried over MgSO₄ and then evaporated. Purification by silica gel chromatography, elution being carried out with CH₂Cl₂, gives the expected product 4.

Method C

A solution of the derivative 4 in CH₂Cl₂ (20 ml/mmol) is treated with BBr₃ (1 eq.) at 0° C. under an argon atmosphere. After stirring for 30 minutes at 0° C., water is added and the solution is filtered so as to give a brown precipitate. The solid product is washed with CH₂Cl₂. Purification on silica gel, elution being carried out with CH₂Cl₂, gives the expected compound 5.

Method D

The 3-amino-5-methoxyphenol 7 (prepared according to Chakraborti, A. K.; Sharma, L.; Nayak, M. K. J. Org, Chem, 2002, 67, 6406-6414) is placed in a round-bottomed flask, with the ethyl acetoacetate derivative 8 (2 to 1.1 eq.) placed in solution in chlorobenzene. The reaction mixture is placed in a microwave reactor. The reaction is carried out at 160° C. and 130 W for a time of between 5 and 30 min.

Observation:

The same reaction can be carried out by thermal heating. The reaction medium is placed directly at 165° C. in a graphite bath. The reaction mixture is left at reflux under argon for between 2 and 72 h. The product of the reaction precipitates from chlorobenzene at ambient temperature, it is filtered and then washed with dichloromethane.

### Physicochemical Characteristics of the Compounds Synthesized According to Methods A, B, C and D

### 3-(3-Chlorophenyl)-5,7-dimethoxy-4-methylquinolin-2(1H)-one, compound I.1

Yield=95%.

¹H NMR (DMSO; 400 MHz) δ11.63 (s, 1H, NH); 7.36 (m, 2H, Ph-Cl); 7.20 (s, 1H, Ph-Cl); 7.10 (m, 1H, Ph-Cl); 6.44 (s, 1H, H₆ or H₈); 6.31 (s, 1H, H₆ or H₈); 3.79 (s, 3H, OCH₃); 3.77 (s, 3H, OCH₃); 2.29 (s, 3H, CH₃).

¹³C NMR (DMSO; 100 MHz) δ: 161.87; 161.24; 160.11; 145.46; 141.83; 139.90; 133.13; 130.95; 130.36; 130.00; 128.25; 127.44; 105.55; 94.48; 91.51; 56.49; 55.91; 22.09.

MS (ESI+) m/z [M+H]⁺ 330, [M+Na]⁺ 352.

### 5,7-Dimethoxy-3-(4-methoxyphenyl)-4-methylquinolin-2(1H)-one, compound I.2

Yield=80%.

¹H NMR (CDCl₃; 400 MHz) δ 12.12 (s, 1H, NH); 7.24 (d, 2H, J=8.8 Hz, Ph-OCH₃); 6.95 (d, 2H, J=8.8 Hz, Ph-OCH₃); 6.37 (d, 1H, J=2.4 Hz, H₆ or H₈); 6.21 (d, 1H, J=2.4 Hz, H₆ or H₈); 3.85 (s, 3H, OCH₃); 3.83 (s, 3H, OCH₃); 3.78 (s, 3H, OCH₃); 2.46 (s, 3H, CH₃).

¹³C NMR (CDCl₃; 100 MHz) δ 163.48 (Cq); 161.43 (Cq); 159.62 (Cq); 158.65 (Cq); 146.86 (Cq); 140.90 (Cq); 131.88 (Ph-OMe C2′ and C6′); 129.15 (Cq); 128.56 (Cq); 113.53 (Ph-OMe C3′ and C5′); 106.83 (Cq); 94.66 (C8); 91.04 (C6); 55.51 (OMe); 55.46 (OMe); 55.30 (OMe); 21.88 (Me).

MS (ESI+) m/z [M+H]⁺ 326, [M+Na]⁺ 348.

### 5,7-Dimethoxy-4-methyl-3-(1-methyl-1H-indol-3-yl)quinolin-2(1H)-one, Compound I.3

Yield=99%.

¹H NMR (DMSO; 400 MHz) δ 11.51 (s, 1H, NH); 7.42 (m, 1H, indol); 7.28 (s, 1H, indol); 7.12 (m, 2H, indol); 6.97 (m, 1H, indol); 6.45 (s, 1H, H₈); 6.31 (s, 1H, H₆); 3.80 (m, 6H, 2 OCH₃ or OCH₃ and CH₃-indol); 3.77 (s, 3H, OCH₃ or CH₃-indol); 2.37 (s, 3H, CH₃).

¹³C NMR (DMSO; 100 MHz) δ 161.33; 160.76; 159.13; 144.77; 140.80; 136.17; 130.43; 127.67; 121.94; 120.75; 119.54; 118.84; 109.71; 108.82; 105.44; 93.63; 90.80; 55.79; 55.24; 32.44; 22.04.

MS (ESI+) m/z [M+H]⁺ 349, [M+Na]⁺ 371.

### 5,7-Dimethoxy-4-methyl-3-(thiophen-2-yl)quinolin-2(1H)-one, Compound I.4

Yield=63%.

¹H NMR (CDCl₃; 400 MHz) δ 12.57 (s, 1H, NH); 7.42 (m, 1H, thiophene); 7.11 (m, 1H, thiophene); 7.02 (m, 1H, thiophene); 6.48 (s, 1H, H₈); 6.23 (s, 1H, H₆); 3.84 (s, 6H, 2 OCH₃); 2.62 (s, 3H, CH₃).

¹³C NMR (CDCl₃; 100 MHz) δ 163.18; 162.14; 159.81; 149.78; 141.17; 137.36; 128.95; 126.51; 126.35; 121.52; 104.10; 95.13; 91.27; 55.68 (2C); 22.42.

MS (ESI+) m/z [M+H]⁺ 302

### 5,7-Dimethoxy-4-methyl-3-phenylquinolin-2(1H)-one, Compound I.5

Yield=98%.

¹H NMR (DMSO; 400 MHz) δ 7.37-7.29 (m, 2H, Ph); 7.29-7.26 (m, 1H, Ph); 7.14-7.12 (m, 2H, Ph); 6.44 (s, 1H, H₈); 6.30 (s, 1H, H₆); 3.78 (s, 3H, OCH₃); 3.76 (s, 3H, OCH₃); 2.28 (s, 3H, CH₃).

¹³C NMR (DMSO; 100 MHz) δ 161.64; 161.51; 160.01; 144.87; 141.71; 137.69; 131.14; 129.71; 128.46; 127.37; 105.65; 94.37; 91.46; 56.44; 55.87; 22.08.

MS (ESI+) m/z [M+H]⁺ 296, [M+Na]⁺ 318.

### 3-(1H-Indol-3-yl)-5,7-dimethoxy-4-methylquinolin-2(1H)-one, Compound I.6

Yield=45%.

¹H NMR (DMSO; 400 MHz) δ 11.52 (s, 1H, NH); 11.19 (s, 1H, NH); 7.41 (m, 1H, indol); 7.30 (s, 1H, indol); 7.14 (m, 1H, indol); 7.08 (m, 1H, indol); 6.97 (m, 1H, indol); 6.49 (s, 1H, H₈); 6.34 (s, 1H, H₆); 3.84 (s, 3H, OCH₃); 3.81 (s, 3H, OCH₃); 2.41 (s, 3H, CH₃).

MS (ESI+) m/z [M+H]⁺ 335, [M+Na]⁺ 357.

### 5-Hydroxy-7-methoxy-4-methyl-3-(thiophen-2-yl)quinolin-2(1H)-one, Compound I.7

Yield=72%.

¹H NMR (DMSO; 400 MHz) δ 11.54 (s, 1H, NH); 10.37 (s, 1H, OH); 7.58 (m, 1H, thiophene); 7.08 (m, 1H, thiophene); 6.96 (m, 1H, thiophene); 6.34 (d, 1H, J=2.4 Hz, H₆ or H₈); 6.22 (d, 1H, J=2.4 Hz, H₆ or H₈); 3.74 (s, 3H, OCH₃); 2.49 (s, 3H, CH₃).

¹³C NMR (DMSO; 100 MHz) δ: 161.39; 160.90; 158.14; 147.84; 141.37; 137.39; 128.79; 126.72; 126.55; 120.92; 104.74; 96.74; 90.20; 55.29; 48.79; 21.90.

MS (ESI+) m/z [M+H]⁺ 288, [M+Na]⁺ 310.

### 5-Hydroxy-7-methoxy-1,4-dimethyl-3-phenylquinolin-2(1H)-one, Compound I.8

Yield=40%.

¹H NMR (DMSO; 400 MHz) δ 7.39-7.37 (m, 2H, Ph); 7.31 (m, 1H, Ph); 7.15 (m, 2H, Ph); 6.43 (s, 1H, H₈ or H₆); 6.35 (s, 1H, H₆ or H₈); 3.83 (s, 3H, OCH₃); 3.56 (s, 3H, CH₃); 2.34 (s, 3H, CH₃).

MS (ESI+) m/z [M+H]⁺ 296, [M+Na]⁺ 318.

### 5-Hydroxy-7-methoxy-4-methyl-3-(naphthalen-2-yl)quinolin-2(1H)-one, Compound I.9

Yield=61%.

¹H NMR (DMSO; 400 MHz) δ 7.92 (m, 3H, biphenyl); 7.72 (s, 1H, biphenyl); 732 (m, 2H, biphenyl); 7.32 (m, 1H, biphenyl) 6.37 (d, 1H, J=2.8 Hz, H₈); 6.24 (d, 1H, J=2.8 Hz, H₆); 3.75 (s, 3H, OCH₃); 2.40 (s, 3H, CH₃).

MS (ESI+) m/z [M+H]⁺ 332, [M+Na]⁺ 354.

### 5,7-Dimethoxy-1,4-dimethyl-3-phenylquinolin-2(1H)-one, Compound I.10

Yield=57%

¹H NMR (DMSO; 400 MHz) δ.7.41-7.37 (m, 2H); 7.33-7.31 (m, 1H); 7.15 (m, 2H); 6.56 (d, J=2 Hz, 1H, H8); 6.48 (d, J=2H, 1H, H6); 3.91 (s, 3H, OCH₃); 3.85 (s, OCH₃); 3.34 (s, 3H, NCH₃), 2.29 (s, 3H, C—CH₃).

¹³C NMR (DMSO; 100 MHz) δ: 161.3; 160.42; 159.86; 142.92; 141.89; 137.84; 130.41 (2C); 128.38; 127.93 (2C); 126.78; 105.64; 93.75; 91.50; 55.96; 55.51; 30.34; 21.79.

MS (ESI⁺) m/z [M+H]⁺ 310, [M+Na]⁺ 332; [M+Na]⁺ ; [2M+Na]⁺ 641.

### 7-Hydroxy-5-methoxy-4-methyl-3-phenylquinolin-2(1H)-one, Compound I.11

Yield=4%.

¹H NMR (DMSO; 400 MHz) δ 7.40-7.36 (m, 2H, Ph); 7.31 (m, 1H, Ph); 7.15 (m, 2H, Ph); 6.35 (s, 1H, H₈); 6.20 (s, 1H, H₆); 3.79 (s, 3H, OCH₃); 2.30 (s, 3H, CH₃).

¹³C NMR (DMSO; 100 MHz) δ: 161.00; 159.52; 144.35; 141.15; 137.27; 130.59; 128.18; 127.81; 126.64; 104.04; 94.41; 93.15; 55.59; 21.45.

MS (ESI+) m/z [M+H]⁺ 282.

### 5-Hydroxy-7-methoxy-4-methyl-3-phenylquinolin-2(1H)-one, Compound I.12

Yield=89%.

¹H NMR (DMSO; 400 MHz) δ 7.39-7.37 (m, 2H, Ph); 7.30 (m, 1H, Ph); 7.16 (m, 2H, Ph); 6.34 (s, 1H, H₈); 6.22 (s, 1H, H₆); 3.74 (s, 3H, OCH₃); 2.36 (s, 3H, CH₃).

¹³C NMR (DMSO; 100 MHz) δ: 161.03; 160.71; 157.82; 144.83; 141.26; 137.15; 130.57; 128.26; 127.81; 126.66; 104.39; 96.33; 90.00; 55.00; 21.22.

MS (ESI+) m/z [M+H]⁺ 282.

### 5,7-Dihydroxy-4-methyl-3-phenylquinolin-2(1H)-one, Compound I.13

Yield=86%.

¹H NMR (DMSO; 400 MHz): δ11.34 (s, 1H, NH); 10.05 (s, 1H, OH); 9.82 (s, 1H, OH); 7.38-7.30 (m, 3H, Ph); 7.15 (m, 2H, Ph); 7.15 (m, 2H, Ph); 6.20 (s, 1H, H₈); 6.12 (s, 1H, H₆); 3.34 (s, 3H, OCH₃); 2.33 (s, 3H, CH₃).

MS (ESI+) m/z [M+H]⁺ 268, [M+Na]⁺ 290.

### 3-Benzyl-5-hydroxy-7-methoxy-4-methylquinolin-2(1H)-one, Compound I.14

Yield=88% (white crystals).

M.p.>278° C., decomposition.

Rf=0.29 (DCM/MeOH 97:3).

¹H NMR (400 MHz, DMSO-d₆): δ 2.53 (s, 3H, Me); 3.72 (s, 3H, OMe); 3.96 (s, 2H, 2×H1′); 6.2 (d, J=2.45 Hz, 1H, H6); 6.34 (d, J=2.45 Hz, 1H, H8); 7.12-7.23 (m, 5H, Ph); 10.23 (s, 1H, OH); 11.45 (s, 1H, NH).

¹³C NMR (100 MHz, DMSO-d₆): δ19.5 (CMe); 31.1 (C1′); 55.0 (COMe); 90.0 (C6); 96.4 (C8); 104.5 (C4a); 125.3 (C3); 125.5 (C5′); 127.9 (C4′, C6′); 128.2 (C3′, C7′) 140.7 (C2′); 140.8 (C8a); 145.3 (C4); 157.4 (C5); 161.3 (C7); 161.9 (C2).

Mass (ESI+): m/z (%) 296 [M+H]⁺ (100), 318 [M+Na]⁺ (50), 613 [2×M+Na]⁺ (30).

### 2,3-Dihydro-9-hydroxy-7-methoxy-1H-cyclopenta[c]quinolin-4(5H)-one, Compound I.15

Yield=74% (brown powder).

M.p. 189-190° C.

Rf=0.2 (DCM/MeOH 97:3).

¹H NMR (400 MHz, DMSO-d₆): δ1.96 (q, J=7.6 Hz, 2H, 2×H2′); 2.6 (t, J=7.6 Hz, 2H, 2×H1′); 3.27 (t, J=7.6 Hz, 2H, 2×H3′); 3.71 (s, 3H, OMe); 6.16 (d, J=2.3 Hz, 1H, H6); 6.33 (d, J=2.3 Hz, 1H, H8); 10.13 (s, 1H, OH); 11.27 (s, 1H, NH).

¹³C NMR (100 MHz, DMSO-d₆): δ 22.6 (C2′); 29.1 (C1′); 35.7 (C3′); 55.0 (COMe); 89.9 (C6); 95.6 (C8); 103.2 (C4a); 128.0 (C3); 142.0 (C8a); 150.6 (C4); 156.1 (C5); 160.6 (C7); 160.7 (C2).

Mass (ESI+): m/z (%) 232 [M+H]⁺ (100), 254 [M+Na]⁺ (50), 485 [2×M+Na]⁺ (60).

### 1-Benzyl-5,7-dimethoxy-4-methyl-3-phenylquinolin-2(1H)-one, Compound I.16

Yield=67%.

¹H NMR (400 MHz, DMSO-d₆): δ 7.45-7.20 (m, 10H); 6.61 (d, J=2 Hz, 1H, H8); 6.50 (d, J=2H, 1H, H6); 4.32 (s, 2H); 3.89 (s, 3H, OCH₃); 3.82 (s, OCH₃); 2.31 (s, 3H, C—CH₃).

Mass (ESI+) m/z [M+H]⁺ 385.

### B. BIOLOGICAL TESTS

B.I Evaluation of the “Potentiating” Effect of the Combination of the Compounds According to the Invention on the Activity of Ciprofloxacin and of their Own Antibiotic Activity

—Compounds

All the compounds according to the invention are solubilized in dimethylsufoxide (DMSO). For each compound, the highest concentration usable during the experiments is determined by taking into account the toxicity of the solvent (final concentration of DMSO in contact with the bacteria of less than 3%) and also the capacity of the compound to solubilize in Mueller Hinton II (MH II) media. This is because some compounds precipitate during dilutions of solutions based on DMSO in MH II media.

—Antibiotic

The antibiotic used is a fluoroquinolone: ciprofloxacin. It is solubilized in sterile osmosed water or MH II acidified with a solution HCl. Twenty μl of 12 N HCl (or 5 μl of 35% HCl) are required to order to solubilize 10 mg of ciprofloxacin in 1 ml of sterile osmosed H₂O.

—Bacterial Strains

The strain for screening the compounds is a strain of Staphylococcus aureus from the American Type Culture Collection or ATCC, called S. aureus ATCC 29213.

—Evaluation of the Potentiating Effect of the Combination of Each Compound According to the Invention on the Activity of Ciprofloxacin

This potentiating effect is evaluated by comparing the MIC of ciprofloxacin alone with that of ciprofloxacin combined with a compound according to the invention according to the MIC method. Said method was carried out according to the protocol described by the Comité de l'Antibiogramme de la Société Française de Microbiologie (CASFM) [Antiobiogram Committee of the French Microbiology Society] and by the Clinical and Laboratory Standards Institute (CLSI). The MICs are determined in a round-bottomed 96-well microplate, in MH II liquid medium. A ciprofloxacin dilution range is prepared in MH II medium. In each well of a first column, 100 μl of a bacterial suspension at 10⁶ CFU/ml, 50 μl of a ciprofloxacin dilution range and 50 μl of MH II are mixed. In each well of a second column, 100 μl of a bacterial suspension at 10⁶ CFU (Colony-Forming Units)/ml, 50 μl of a ciprofloxacin concentration range and 50 μl of a solution of a compound according to the invention at the highest possible concentration are mixed. After 18-24 h of incubation at 37° C., the lowest concentration of antibiotic for which no bacteria growth is observed (MIC) is noted in the two columns with and without synthetic molecule. A compound according to the invention has a potentiating effect on the activity of ciprofloxacin compared with the effect of ciprofloxacin alone if the MIC thereof is decreased by a dilution factor 4 in the presence of this compound.

—Evaluation of the Antibiotic Activity of the Compounds According to the Invention Alone

The antibiotic activity of a molecule is evaluated using the MIC technique. The MIC of a molecule is determined in a 96-well microplate, in MH II liquid medium according to the protocol of the CASFM and of the CLSI. A dilution range of the molecule is prepared beforehand in MH II medium. In each well, 100 μl of a bacterial suspension of SI aureus ATCC 29213 (10⁶ CFU/ml), 50 μl of the dilution range of the compound tested and 50 μl of MH II are mixed. After 18-24 h of incubation at 37° C., the molecules having shown an inhibition of bacterial growth are recorded as having an antibiotic activity. The lowest concentration of compound for which no bacterial growth is observed is the MIC (lowest concentration inhibiting the growth of the bacteria).

—Results

The 16 compounds according to the invention that were tested potentiate the activity of ciprofloxacin (to different degrees).

Among the 16 compounds, 4 compounds (the compounds I.11, I.12, I.13 and I.7) also exhibit an antibiotic activity alone. The compounds I.12, I.13 and I.7 show an antibiotic activity for concentrations of, respectively, 62.5-125 μM, 155 μM and 62.5-125 μM. The compound I.11 is not present in sufficient amount to continue the analyses.

B.II Description of the Spectrum of Activity of the Compound I.12

—Antibiotic and Compound I.12

The antibiotic used is ciprofloxacin with the same dilution as in section B.1. The compound I.12 is also used under the same conditions as in section B.1.

—Bacterial Strains

Fifty-six strains belonging to 19 genera were used (TABLE 6).

TABLE 6

| | | | Resistances | Strains |
|---|---|---|---|---|
| Staphylococcus | | aureus | — | ATCC 29 213 |
| | | | — | ATCC 25 923 |
| | | | MRSA | ST07 1012 |
| | | | MRSA | HT06 0164 |
| | | | MRSA | HT02 0634 |
| | | | MRSA | HT03 0336 |
| | | | MRSA | HT04 0473 |
| | | | MRSA | HT05 0109 |
| | | | MRSA | ST07 1170 |
| | | | MRSA | HT03 0870 |
| | | | MRSA | HT02 0290 |
| | | | MRSA | HT06 0163 |
| | | | VISA | V1 |
| | | | VISA | V3 |
| | | | VISA | V4 |
| | | | VISA | V5 |
| | | | VISA | V7 |
| | | epidermidis | — | ATCC 14 990 |
| | | | | (CIP 81551) |
| Enterococcus | | faecium | VRE VanA | 1 |
| | | | VRE VanA | 2 |
| | | | VRE VanA | 3 |
| | | | VRE VanB | 4 |
| | | | VRE VanB | 5 |
| | | | VRE VanB | 6 |
| | | | — | B509081205 |
| | | | VRE VanA | 8196211 |
| | | | VRE VanB | 8445271 |
| | | faecalis | — | ATCC 29212 |
| | | | VRE VanA | 7 |
| | | | VRE VanA | 8 |
| | | | VRE VanA | 9 |
| | | | VRE VanB | 10 |
| | | | VRE VanB | 11 |
| | | | VRE VanB | 12 |
| Streptococcus | | pneumoniae | — | ATCC 49619 |
| | | | | (CIP 104485) |
| | | agalactiae (Strepto | — | (Mar. 10, 2008) |
| | | B) | | |
| | | pyogenes (Strepto | — | 8370 |
| | | A) | | (Jan. 15, 2009) |
| Listeria | | monocytogenes | — | 4243118 |
| Bacillus | | cereus | — | 17.1240 |
| | | subtilis | — | ATCC 6683 |
| Corynebacterium | | amycolatum | — | 103452T |
| Acinetobacter | | baumannii | — | Strain 1 |
| Pseudomonas | | aeruginosa | — | 15 442 |
| | | fluorescens | — | OO25577401 |
| Burkholderia | | cepacia | — | 734 |
| Stenotrophomonas | | maltophilia | — | ATCC 17666 |
| enterobacteriaceae | Escherichia | coli | — | ATCC 25922 |
| | Klebsiella | pneumoniae | — | 26.2362 |
| | | oxytoca | — | ATCC 700324 |
| | Salmonella | enterica | — | HH 0436412 |
| | Citrobacter | freundii | — | 26.7009 |
| | Enterobacter | aerogenes | — | — |
| | | sakazakii | — | ATCC 51329 |
| | Serratia | marscecens | — | 40437322 |
| | | | | (Jan. 14, 2009) |
| | Proteus | mirabilis | — | 26.5354 |
| | Yersinia | enterocolitica | — | O8484255 |

Nine of these strains are ATCC strains and 47 are of clinical origins and come from the Centre de Biologie Est [Eastern Biological Center] in Bron. Eighteen staphylococcal strains are tested: 1 strain of S. epidermidis, 2 strains of S. aureus, 10 MRSA (methicillin-resistant S. aureus) and 5 VISA (vancomycin intermediate sensitivity S. aureus). Sixteen enterococcal strains are also tested, i.e. 9 E. faecium and 7 E. faecalis. The latter 2 species grouped together 2 vancomycin-sensitive strains (1 per species), and 14 resistant strains (VRE, respectively 8 E. faecium and 6 E. faecalis). The other genera are listed in TABLE 6 above.

—Evaluation of the Potentiating Effect of the Combination of the Compound I.12 on the Activity of Ciprofloxacin

This potentiating effect is evaluated as previously. However, the growth of certain microorganisms (Streptococcus pneumoniae, S. agalactiae, S. pyogenes, Corynebacterium amycolatum and Listeria monocytogenes) in liquid medium requires defibrinated horse blood lysed in 50% of sterile osmosed water via a succession of 7 freezings (−20° C.)/thawing (ambient temperature). The 50% blood is then centrifuged at ambient temperature at 12 000 rpm for 20 min. The supernatant is introduced into sterile, additive-free MH II in an amount of 5% by volume.

—Evaluation of the Antibiotic Activity of the Compound I.12

The antibiotic activity of the compound I.12 is evaluated using the MIC technique according to the protocol described previously, taking into account the blood requirements of certain strains.

—Results

Among the 56 bacterial strains tested, 28 were found to be more sensitive to ciprofloxacin when the compound I.12 is combined therewith. These strains are gram-positive bacteria of staphylococcal and enterococcal type including resistant strains (or even multiresistant strains):
2/2 S. aureus sensitive to conventional antibiotics, 10/10 methicillin-resistant S. aureus (MRSA) and 5/5 S. aureus with reduced sensitivity to vancomycin (VISA);
1/1 S. epidermidis.
5/7 E. faecalis (4 are VREs, i.e. vancomycin-resistant strains) and 5/9 E. faecium (the 5 being VREs).

TABLE 7 shows the distribution of the staphylococcal and enterococcal strains sensitive to the combination of ciprofloxacin and the compound I.12.

TABLE 7

| | | | | | Enterococcus | | | |
|---|---|---|---|---|---|---|---|---|
| Staphylococcus | | | | | faecalis | | faecium | |
| | aureus (n = 17) | | | epidermidis (n = 1) | (n = 7) | | (n = 9) | |
| Resistances | MSSA | MRSA | VISA | Sensitive strain | VSE | VRE | VSE | VRE |
| Number of strains tested | 2 | 10 | 5 | 1 | 1 | 6 | 1 | 8 |
| Number of sensitive strains | 2 | 10 | 5 | 1 | 1 | 4 | 0 | 5 |
| Percentage | 100% | 100% | 100% | 100% | 100% | 67% | 0 | 62.5% |

MSSA: Methicillin-sensitive S. aureus;
MRSA: methicillin-resistant S. aureus;
VISA: vancomycin intermediate sensitivity S. aureus;
VSE: vancomycin-sensitive enterococcus;
VRE: vancomycin-resistant enterococcus

For each strain, the ciprofloxacin MICs are divided by a factor 16 in the presence of the compound I.12, with the exception of one strain, the activity of which is ≧4. Two E. faecium VRE strains not listed in the 28 previously mentioned are weakly sensitive to the action of the combination of the compound I.12 with ciprofloxacin (factor ≧2).

The strains sensitive to the action of the compound I.12 alone are the same strains (the compound I.12's own antibiotic effect).

B-III—Investigation of the Mode of Action of the Compound I.12

—Antibiotics

The antibiotics used are erythromycin, ciprofloxacin, vancomycin, tetracycline and oxacillin.

The ciprofloxacin is prepared as described previously.

The erythromycin (storage at 4° C.) is taken up in 96% ethanol at a concentration of 10 mg/ml. The solution is then diluted 10-fold and then 31.3-fold in MH II broth in order to obtain a stock solution of 32 μg/ml (8 μg/ml in the well).

The vancomycin is taken up at a concentration of 10 mg/ml of sterile osmosed water. The solution is subsequently diluted 10-fold and then 15.6-fold in MH II broth in order to obtain a stock solution of 64 μg/ml (16 μg/ml in the well).

The tetracycline is taken up in sterile osmosed water at a concentration of 10 mg/ml of tetracycline. The solution is subsequently diluted 10-fold and then 31.3-fold in MH II broth in order to obtain a stock solution of 32 μg/ml (8 μg/ml in the well).

The oxacillin is taken up in sterile osmosed water at the concentration of 10 mg/ml. The solution is subsequently diluted 10-fold and then 62.5-fold in MH II broth so as to obtain a stock solution of 16 μg/ml (4 μg/ml in the well).

—Bacterial Strains

The S. aureus strains used are:
the reference strain ATCC 29213;
two strains ST07 1012 and V4 identified as, respectively, methicillin-resistant (MRSA) and vancomycin-resistant (VISA);
genetically modified strains and the strains from which they are derived: S. aureus 1199b (overexpressing the NorA efflux pump) and its “parent” strain 1199; S. aureus K 1712 (exhibiting no NorA efflux pump at the level of its bacterial membrane) and its “parent” strain 8325-4.

—Evaluation of the Antibacterial Activity of the Combination of The Compound I.12 with Various Antibiotics on the S. Aureus ATCC 29213 Strain (Chessboard Method)

The sensitivity of S. aureus ATCC 29213 to the combination of the antibiotic molecules ciprofloxacin and the compound I.12 and the effect of each of the two molecules on the activity of the other are determined using the chessboard method. This method is carried out in a round-bottomed 96-well plate (12 columns by 8 rows). Various concentrations of ciprofloxacin and of compound I.12 are obtained by a dilution of their stock solutions in MH II broth. Fifty microliters of the dilution range of the compound I.12 are deposited in each well of columns 1 to 10, each row corresponding to a dilution of this compound. Fifty microliters of the dilution range of ciprofloxacin are deposited in the wells of columns 2 to 10, each column corresponding to a dilution. This same dilution range of ciprofloxacin is also prepared in column 11. Fifty microliters of MH II broth are deposited in the wells of columns 1 and 11. Finally, 100 μl of bacterial suspension at 10⁶ CFU (Colony-Forming Units)/ml are introduced in each well of columns 1 to 10 (final concentration of bacteria: 5×10⁵ CFU/ml).

The plate is read visually. The 200 μl of reagents introduced into each well are either cloudy (bacterial growth) or translucent (absence of bacterial growth). The fractional inhibitory concentration (FIC) is calculated by adding the FIC of the compound I.12 (FIC_{I.12}) and the FIC of ciprofloxacin (FIC_{cipro}) according to the following formulae:

FIC_{I.12}=MIC of the compound I.12 in combination with ciprofloxacin/MIC of the compound I.12 alone

FIC_{cipro}=MIC of the compound I.12 in combination with ciprofloxacin/MIC of ciprofloxacin alone

FIC=FIC_{I.12}+FIC_{cipro}

The interpretation of the results is the following: an FIC≦0.5 corresponds to a synergy between the two molecules, an FIC>4 corresponds to an antagonism of the two molecules and an FIC of between 0.5 and 4 corresponds to an absence of interaction between the two molecules.

—Evaluation of the Antibacterial Activity of the I.12/Ciprofloxacin Combination on the S. Aureus ATCC 29213 Strain (Time Kill Curve Method)

The time kill curve is performed in 6-well plates. Each well contains 5 ml of MH II broth inoculated with bacteria in the exponential growth phase (final concentrations at 10⁶ CFU/ml). The various bacterial growth conditions tested are:
Well 1: 0.5 mg/ml of ciprofloxacin +31 μM of I.12.
Well 2: 0.125 mg/ml of ciprofloxacin +31 μM of I.12.
Well 3: 0.5 mg/ml of ciprofloxacin.
Well 4: 0.125 mg/ml of ciprofloxacin.
Well 5: 31 μM of I.12.
Well 6: No molecule.

The cultures are incubated at 37° C. with shaking at 400 rpm. Samples are taken every hour for the first 6 hours, and at 22 hours. The live bacteria are counted by culturing on a dish after dilutions of the cultures. A synergistic or antagonist effect of the two molecules is defined by a decrease ≧2 log₁₀ CFU/ml and an increase ≧2 log₁₀ CFU/ml between the wells containing the combination of the two molecules and those containing the most active of the molecules.

—Results

—Evaluation of the Antibacterial Activity of the Combination of the Compound I.12 with Various Antibiotics on the S. Aureus ATCC 29213 Strain (Chessboard Method)

The chessboard method demonstrates:
A synergistic (FIC≦0.5) antibacterial combination of I.12 and ciprofloxacin on S. aureus ATCC 29213 or resistant strains of MRSA (strain ST07 1012) and VISA (strain V4) type. An absence of interaction (FIC>0.5 but <4) between I.12 and oxacillin, erythromycin, tetracycline or vancomycin on the same bacterial strain.
An absence of interaction for each ATB with the K 1712 strain (strain without NorA pump) and a highly synergistic combination with the 1199B strain (strain overexpressing NorA).

—Evaluation of the Antibacterial Activity of the I.12/Ciprofloxacin Combination on the S. Aureus a TCC 29213 Strain (Time Kill Curve Method)

The results are a mean of two experiments. A synergistic effect is observed with the ciprofloxacin (0.5 mg/ml and 0.125 mg/m)/I.12 (31 μM) combination (reduction ≧2 log₁₀) as shown in the single FIGURE which presents the evaluation of antibacterial activity of the I.12/ciprofloxacin combination using the time kill curve method.

In conclusion, the compounds according to the invention, in combination with ciprofloxacin, clearly promote the activity of this antibiotic on gram-positive bacteria of the staphylococcus and enterococcus genera, in particular of the resistant strains (MRSA, VISA, VRE), which are the scourge of hospitals.

Some of these compounds also have a notable antibiotic activity. In combination with ciprofloxacin, their action becomes synergistic with that of the antibiotic (and not simply additive). This synergy makes it possible to reduce the antibiotic concentrations used in vitro for destroying bacteria, and probably those used in vivo (reduction in the toxic effects attributed to the anti-infective molecules). This activity is found including on MRSA and VISA strains. The presence of a synergistic effect is probably linked to the efflux-pump-inhibiting activity of the compounds according to the invention.

This synergistic effect is present only for antibiotics of fluoroquinolone type, in particular hydrophilic fluoroquinolones such as nofloxacin, ciprofloxacin, etc. This activity is excellent for the 1199B strain (overexpressing NorA), but is virtually zero for the K 1712 strain (devoid of NorA pump). This tends to indicate an inhibitory action on bacterial efflux pumps, in particular NorA pumps.

B-IV. Supplemental Tests Demonstrating the Inhibitory Activity of the Compounds According to the Invention with Respect to NorA

—Compounds

The molecules of which the EPI effect was evaluated are: I.1 (24 μmol/l), I.6 (16 μmol/l) and I.14 (31 μmol/l).

—Antibiotics

The antibiotics with which these molecules were combined are: ciprofloxacin and norfloxacin; tetracycline; oxacillin; erythromycin and vancomycin.

Bacterial Strains.

The bacterial strains tested are S. aureusATCC 29213; S. aureus 1199b overexpressing NorA and also S. aureus 1199, the strain from which it is derived; S. aureus K1712 not expressing NorA and also S. aureus 8325.4 (the strain from which it is derived).

—Evaluation of the Potentiating Effect of the Combination of Each Compound According to the Invention Above on the Activity of Ciprofloxacin

The methodology used is based on the MIC technique described previously. Only the combinations of molecules of which the activities comply with the condition “MIC_{antibiotic}/MIC_{antibiotic+compound according to the invention}≧4” were retained as demonstrating an inhibitory activity on the NorA efflux pump.

The results were the following:
The compound I.6 enables a 4-fold, 8-fold or even 16-fold improvement in the MICs when it is in the presence of norfloxacin and of ciprofloxacin. This activity is particularly notable when the compound combined with one of the two fluoroquinolones is evaluated with the 1199b strain overexpressing NorA. However, this activity is zero when the combinations are evaluated on the K1712 strain. Finally, this activity is not very effective when the molecule is coupled to non-fluoroquinolone antibiotics.
The results are identical for the compound I.14. A weak activity which does not exist for the compound I.6 is, however, noted on the 1199b strain when the EPI molecule is in the presence of tetracycline and of erythromycin.
Effects of the compound I.1 appear to exist, but they are minor.

These compounds according to the invention preferentially show an activity in combination with fluoroquinolones. Furthermore, among all the strains, S. aureus 1199b is the strain which is most sensitive to the activity of the combination, unlike the K1712 strain for which the combination did not promote the activity of the conventional antibiotic.

All of these results point toward a potentiating activity of I.6 and I.14 when these compounds are in combination with fluoroquinolones, molecules released out of the bacterium by NorA. Furthermore, the strain overexpressing NorA (1199b) is very sensitive to this combination, whereas the one which no longer expresses it (K1712) is, on the contrary, not very sensitive at all. The target of the compounds according to the invention clearly appears to be NorA.

1-21. (canceled)

22. A method for taking a prophylactic therapeutic measure against a disease or disorder or treating the disease or the disorder by administration of a medicament containing an antimicrobial agent in a subject in need thereof, said method comprising the administration of a compounds of formula (I): 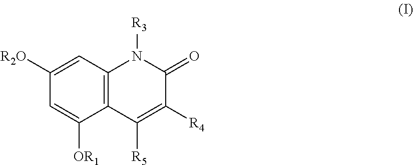
in which:
R₁ and R₂, which may be identical or different, are each independently a hydrogen atom or an unsubstituted or substituted (C₁-C₁₂)alkyl group,
R₃ is a hydrogen atom or an unsubstituted or substituted (C₁-C₆)alkyl group, or an unsubstituted or substituted benzyl group,
R₄ is an unsubstituted or substituted (C₁-C₁₂)alkyl group, an aryl group or a heteroaryl group, it being possible for said aryl and heteroaryl groups to be unsubstituted or substituted, and R₅ is an unsubstituted or substituted (C₁-C₁₂)alkyl group,
or else R₄ and R₅ are linked to one another by a saturated hydrocarbon chain containing 3 or 4 carbon atoms,
optionally in hydrated form or in the form of a salt which is acceptable for administration to animals or plants,
as the antimicrobial agent or as a potentiator of the effect of the antimicrobial agent.

23. The method as claimed in claim 22, characterized in that R₃ is a hydrogen atom or a methyl or benzyl group.

24. The method as claimed in claim 22, characterized in that R₁ and R₂, which may be identical or different, are each independently a hydrogen atom or a methyl group.

25. The method as claimed in claim 22, characterized in that R₅ is a methyl group.

26. The method as claimed in claim 22, characterized in that R₄ is a benzyl, phenyl, naphthyl, thiophenyl and indolyl group, it being possible for said groups to be unsubstituted or substituted with one or more constituents chosen from chlorine, bromine, iodine and fluorine atoms, and (C₁-C₆)alkyl and (C₁-C₆)alkoxy groups.

27. The method as claimed in claim 22, characterized in that the administered compound is chosen from:
3-(3-chlorophenyl)-5,7-dimethoxy-4-methylquinolin-2(1H)-one, compound I.1,
5,7-dimethoxy-3-(4-methoxyphenyl)-4-methylquinolin-2(1H)-one, compound I.2,
5,7-dimethoxy-4-methyl-3-(1-methyl-1H-indol-3-yl)quinolin-2(1H)-one, compound I.3,
5,7-dimethoxy-4-methyl-3-(thiophen-2-yl)quinolin-2(1H)-one, compound I.4,
5,7-dimethoxy-4-methyl-3-phenylquinolin-2(1H)-one, compound I.5,
3-(1H-indol-3-yl)-5,7-dimethoxy-4-methylquinolin-2(1H)-one, compound I.6,
5-hydroxy-7-methoxy-4-methyl-3-(thiophen-2-yl)quinolin-2(1H)-one, compound I.7,
5-hydroxy-7-methoxy-1,4-dimethyl-3-phenylquinolin-2(1H)-one, compound I.8,
5-hydroxy-7-methoxy-4-methyl-3-(naphthalen-2-yl)quinolin-2(1H)-one, compound I.9,
5,7-dimethoxy-1,4-dimethyl-3-phenylquinolin-2(1H)-one, compound I.10,
7-hydroxy-5-methoxy-4-methyl-3-phenylquinolin-2(1H)-one, compound I.11,
5-hydroxy-7-methoxy-4-methyl-3-phenylquinolin-2(1H)-one, compound I.12,
5,7-dihydroxy-4-methyl-3-phenylquinolin-2(1H)-one, compound I.13,
3-benzyl-5-hydroxy-7-methoxy-4-methylquinolin-2(1H)-one, compound I.14,
2,3-dihydro-9-hydroxy-7-methoxy-1H-cyclopenta[c]quinolin-4(5H)-one, compound I.15,
1-benzyl-5,7-dimethoxy-4-methyl-3-phenylquinolin-2(1H)-one, compound I.16,
optionally in hydrated form or in the form of a salt which is acceptable for administration to animals or plants.

28. The method as claimed in claim 22, characterized in that the compound is administered as a potentiator of the effect of an antimicrobial agent such as an antibiotic or an antiseptic, to which bacteria are resistant through expulsion via an efflux pump, and in particular the NorA pump.

29. The method as claimed in claim 28, characterized in that the bacteria are bacteria of gram-positive cocci type advantageously chosen from: Enterococcus, such as Enterococcus faecalis and Enterococcus faecium; Staphylococcus, such as Staphylococcus aureus and Staphylococcus epidermis.

30. The method as claimed in claim 28, characterized in that the compound is administered as a potentiator of the effect of an antimicrobial agent which is an antibiotic.

31. The method as claimed in claim 30, characterized in that the antibiotic agent is chosen from: tetracyclines, macrolides, ansamycins, β-lactam antibiotics and, preferably, fluoroquinolones chosen from enofloxacin, ofloxacin, levofloxacin, moxifloxacin and, preferentially, ciprofloxacin and norfloxacin.

32. Compounds chosen from:
3-(3-chlorophenyl)-5,7-dimethoxy-4-methylquinolin-2(1H)-one, compound I.1,
5,7-dimethoxy-3-(4-methoxyphenyl)-4-methylquinolin-2(1H)-one, compound I.2,
5,7-dimethoxy-4-methyl-3-(1-methyl-1H-indol-3-yl)quinolin-2(1H)-one, compound I.3,
5,7-dimethoxy-4-methyl-3-(thiophen-2-yl)quinolin-2(1H)-one, compound I.4,
5,7-dimethoxy-4-methyl-3-phenylquinolin-2(1H)-one, compound I.5,
3-(1H-indol-3-yl)-5,7-dimethoxy-4-methylquinolin-2(1H)-one, compound I.6,
5-hydroxy-7-methoxy-4-methyl-3-(thiophen-2-yl)quinolin-2(1H)-one, compound I.7,
5-hydroxy-7-methoxy-1,4-dimethyl-3-phenylquinolin-2(1H)-one, compound I.8,
5-hydroxy-7-methoxy-4-methyl-3-(naphthalen-2-yl)quinolin-2(1H)-one, compound I.9,
5,7-dimethoxy-1,4-dimethyl-3-phenylquinolin-2(1H)-one, compound I.10,
7-hydroxy-5-methoxy-4-methyl-3-phenylquinolin-2(1H)-one, compound I.11,
5-hydroxy-7-methoxy-4-methyl-3-phenylquinolin-2(1H)-one, compound I.12,
5,7-dihydroxy-4-methyl-3-phenylquinolin-2(1H)-one, compound I.13,
3-benzyl-5-hydroxy-7-methoxy-4-methylquinolin-2(1H)-one, compound I.14,
2,3-dihydro-9-hydroxy-7-methoxy-1H-cyclopenta[c]quinolin-4(5H)-one, compound I.15,
1-benzyl-5,7-dimethoxy-4-methyl-3-phenylquinolin-2(1H)-one, compound I.16,
optionally in hydrated form or in the form of a salt which is acceptable for administration to animals or plants.

33. The compound of the formula (Ip): 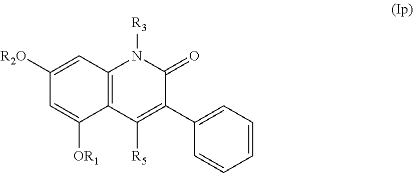
in which:
R₁ and R₂, which may be identical or different, are each independently a hydrogen atom or an unsubstituted or substituted (C₁-C₁₂)alkyl group,
R₃ is a hydrogen atom or an unsubstituted or substituted (C₁-C₆)alkyl group, or an unsubstituted or substituted benzyl group,
R₅ is an unsubstituted or substituted (C₁-C₁₂)alkyl group, optionally in hydrated form or in the form of a salt which is acceptable for administration to animals or plants.

34. The compound as claimed in claim 33, characterized in that R₅ is a methyl group.

35. The compound as claimed in claim 33, characterized in that R₃ is a hydrogen atom or a methyl or benzyl group.

36. The compound as claimed in claim 33, characterized in that R₁ and R₂, which may be identical or different, are each independently a hydrogen atom or a methyl group.

37. The compound as claimed in claim 33, characterized in that R₅=Me, R₁=H and R₂=Me.

38. A pharmaceutical composition containing a compound as claimed in claim 22, for use thereof as a potentiator of the effect of an antimicrobial agent, or said compound in combination with at least one pharmaceutically acceptable excipient, characterized in that it also contains an antimicrobial agent, the effect of which is to be potentiated by said compound.

39. The pharmaceutical composition containing a compound as claimed in claim 32, in combination with at least one pharmaceutically acceptable excipient.

40. A method of demonstrating, in vitro, a presence of bacteria resistant to a given antibiotic sample or demonstrating a degree of resistance to an antibiotic of bacteria present in a biological sample by using the compound of claim 22.

41. A method of demonstrating, in vitro, a presence of bacteria resistant to a given antibiotic sample or demonstrating a degree of resistance to an antibiotic of bacteria present in a biological sample by using one of the compounds of claim 32.

42. A method of demonstrating, in vitro, a presence of bacteria resistant to a given antibiotic sample or demonstrating a degree of resistance to an antibiotic of bacteria present in a biological sample by using the compound of claim 33.

43. A method for taking a prophylactic therapeutic measure against a disease or disorder or treating the disease or the disorder r by administration of a medicament in a subject in need thereof, said method comprising the administration of the compound of claim 33 as the medicament or as a medicament in combination with an antimicrobial agent to potentiate an effect of the antimicrobial agent.

44. Pharmaceutical compositions containing a compound as claimed in claim 33, in combination with at least one pharmaceutically acceptable excipient.
